(19) United States
(12) Patent Application Publication
(10) Pub. No.: US 2024/0408043 A1
(43) Pub. Date: Dec. 12, 2024

(54) COMPOSITIONS AND METHODS FOR USE IN INCREASING MOTIVATIONAL PERFORMANCE

(71) Applicant: SOCIETE DES PRODUITS NESTLE S.A., Vevey (CH)

(72) Inventors: FREDERIC RONGA, St-Cierges (CH); GUILLAUME JACOT, Lausanne (CH); TERESA MENDES, Pully (CH); LOUISE BAUR, Lausanne (CH); INDIA C. SEVERIN, Lausanne (CH); CARMEN SANDI, Bremblens (CH)

(21) Appl. No.: 18/262,762

(22) PCT Filed: Jan. 27, 2022
(86) PCT No.: PCT/EP2022/051834

§ 371 Date: Jul. 25, 2023

(30) Foreign Application Priority Data
Jan. 27, 2021 (FR) FR2100776

(51) Int. Cl. A61K 31/198; A61K 31/4415; A61K 31/714; A61P 25/28

(52) U.S. Cl. CPC A61K 31/198; A61K 31/4415; A61K 31/714; A61P 25/28

(57) ABSTRACT

The present invention relates to compositions comprising N-acetylcysteine and B vitamins for increasing motivational performance and/or mental energy. The present invention further relates to such compositions provided as a kit together with a digital tool such as a companion App.

### FIELD OF THE INVENTION

The present invention relates to compositions comprising N-acetylcysteine and B vitamins for increasing motivational performance and/or mental energy. The present invention further relates to such compositions provided as a kit together with a digital tool such as a companion App.

### BACKGROUND TO THE INVENTION

Glutathione (GSH) is an essential antioxidant used by the body to prevent cellular and tissue damage. It is involved in many fundamental metabolic processes ranging from the nitric oxide cycle to dietary mineral incorporation. Additionally, glutathione is instrumental for cells to regulate their division and their differentiation from progenitor cells into mature somatic cells.

As one of the body's core antioxidants, glutathione binds circulating reactive oxygen species (ROS) which can cause cellular and DNA damage if left unchecked. Reactive oxygen species, also known as free radicals, are byproducts of metabolism and can be broadly harmful to the body. To scavenge circulating ROS, glutathione binds to ROS thereby becoming oxidized. This means that glutathione prevents important cellular proteins or DNA from being oxidized, which can inhibit their function.

Increased oxidative damage and decreased glutathione levels are observed under situations of high energetic demand in the brain such as psychogenic stress.

High concentrations of oxidized glutathione in the brain are a hallmark that the brain is in a compromised state, but high concentrations in the blood plasma may be considered to be healthy and normal. The reason is that oxidized glutathione must return to the bloodstream from the brain in order to discharge the ROS it carries into a metabolic processes which can make use of them constructively. Alternatively, the oxidized form of GSH can be locally reverted back into the reduced state by glutathione reductase or it can return from the brain to the bloodstream in order to discharge the ROS constructively. As such, high concentrations of oxidized glutathione in the brain may mean that there is not enough glutathione to remove all of the reactive oxygen species that are circulating, indicating severe levels of stress.

By only measuring glutathione levels in blood plasma, one may erroneously assume that circulating glutathione is normal, even in cognitively impaired individuals. Only recently, it was recognized that cognitively impaired individuals have decreased glutathione levels in the brain, however, it is not known under what conditions glutathione levels in the brain may transiently change in normal healthy individuals related to their performance of different cognitive and motor tasks.

Direct supplementation with GSH is a challenging approach, particularly because it can be rapidly degraded in the liver into its constituent amino acids as well as be partially hydrolyzed and oxidized. Consequently, GSH bioavailability is limited following oral administration. Therefore, there is a need to find compositions and methods of increasing glutathione in the brain in situations of high energetic demand in the brain.

### SUMMARY OF THE INVENTION

The present invention provides compositions and methods for enhancement of glutathione levels in the brain. In particular, the present invention provides compositions comprising at least one N-Acetylcystein or functional derivative and at least one B-vitamin. The present composition is for use in increasing glutathione levels in the brain and/or increasing motivational performance or mental energy in an individual.

As mentioned above, direct oral administration of glutathione has limited bioavailable effectiveness. The present invention provides solutions for enhancement of glutathione in the brain, particularly in the nucleus accumbens region, during high energy demands in the brain.

In a preferred embodiment, the composition is in the form of a food supplement or dietary supplement, such as a gummy, powder sachet or tablet.

In a preferred embodiment, Vitamins are B Vitamins, such as B6, B12.

In another embodiment, the present invention relates to a kit comprising a composition as described above and a digital tool.

In a preferred embodiment, the digital tool is a companion App.

In another embodiment, the composition and the companion App helps the individual to achieve objectives and/or to support his motivation; reduces his perception of effort to reach his objectives.

### DESCRIPTION OF FIGURES

FIG. 1—Enhancement of GSH via systemic treatment with N-acetyl cysteine (NAC) results in increased motivational performance in adult male rats

(A) Schematic for training schedule—Rats were trained to nose poke for saccharine pellets on an FR1 schedule (1 nose poke gives 1 pellet). They were then treated with either NAC (N-Acetylcysteine) or vehicle for 2 weeks in the drinking water. They were given a reminder training session and then 24 h later tested for their motivated effort in a progressive ratio task.

(B) NAC treated rats showed enhanced GSH in the nucleus accumbens compared to vehicle treated rats

(C) NAC treated rats made significantly more nose pokes for the reward than vehicle treated rats

(D) NAC treated rats earned more overall rewards than vehicle treated rats

(E) NAC treated rats exhibited a higher breakpoint than vehicle treated rats.

### DETAILED DESCRIPTION OF THE INVENTION

### Definitions

All percentages are by weight of the total weight of the composition unless expressed otherwise. Similarly, all amounts and all ratios are by weight unless expressed otherwise. When reference is made to the pH, values correspond to pH measured at 25° C. with standard equipment. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms.

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

The “subject” or “individual” of the present invention is a human adult subject, preferably a healthy adult with the need to improve motivational performance through modulating glutathione levels in the brain. The compositions of the invention may be beneficially used for increasing glutathione level in the brain, in particular, the nucleus accumbens for preventing or treating conditions or diseases which are characterized by low glutathione levels in the brain, whether transient or chronic.

In biology and psychology, the term “stress” refers to the consequence of the failure of a human or other animal to respond appropriately to physiological, emotional, or physical threats, whether actual or imagined. The psychobiological features of stress may present as manifestations of oxidative stress, i.e., an imbalance between the production and manifestation of reactive oxygen species and the ability of a biological system readily to detoxify the reactive intermediates or to repair the resulting damage. Disturbances in the normal redox state of tissues can cause toxic effects through the production of peroxides and free radicals that damage all of the components of the cell, including proteins, lipids, and DNA. Some reactive oxidative species can even act as messengers through a phenomenon called “redox signaling.”

“Reactive oxygen species” play important roles in cell signaling, a process termed redox signaling. Thus, to maintain proper cellular homeostasis a balance must be struck between reactive oxygen production and consumption. One source of reactive oxygen under normal conditions in humans is the leakage of activated oxygen from mitochondria during oxidative phosphorylation. Other enzymes capable of producing superoxide (O2−) are xanthine oxidase, NADPH oxidases and cytochromes P450. Hydrogen peroxide, another strong oxidizing agent, is produced by a wide variety of enzymes including several oxidases.

The terms “treatment” and “treating” include any effect that results in the improvement of the condition or disorder, for example lessening, reducing, modulating, or eliminating the condition or disorder. The term does not necessarily imply that a subject is treated until total recovery. Non-limiting examples of “treating” or “treatment of” a condition or disorder include: (1) inhibiting the condition or disorder, i.e., arresting the development of the condition or disorder or its clinical symptoms and (2) relieving the condition or disorder, i.e., causing the temporary or permanent regression of the condition or disorder or its clinical symptoms. A treatment can be patient- or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. The terms “condition” and “disorder” mean any disease, condition, symptom, or indication.

The relative terms “improved,” “increased,” “enhanced” and the like refer to the effects of the composition on increasing glutathione in the brain, in particular in the nucleus accumbens region of the brain, and subsequently improving the cognitive or motor performance in the individual subject.

“Motivational performance” is synonymous with the terms “mental energy” and related terms of “volition”, “will-power”, “time-on-task”, “persistence”, “self-control”, “sustained effort”, and “self-efficacy”. All these terms relate to a person's drive to initiate and do things. Motivational performance is linked to subjectively perceived self-efficacy and well-being.

Motivational performance describes the subjective perception of mental resources available, which in turn is linked to cognitive functioning (Egan et al. (2015) Personality & Social Psychology Bulletin, 41(3), 336-350). For example, motivational performance is reduced in states of depression and anxiety (O'Connor et al. (2006) Nutrition Reviews, 64 (7 Pt 2), S2-6).

Measurement of “motivational performance” can be by both motor tasks and cognitive tasks. Typically, these motor tasks and cognitive tasks are performed under incentivized conditions, meaning that individuals get an incentive depending on their performance of the task.

For example, a motor task under incentivized conditions may be measured as an individual's ability to perform a strenuous motor task, e.g. squeezing a handgrip measuring both force and endurance wherein the performance is normalised for individual muscular strength (Zhu et al. (2019) NeuroImage. Clinical, 23, 101922).

For example, a cognitive task under incentivized conditions may be an individual's ability to perform a strenuous cognitive task, e.g. continuous/sustained attention and working memory (e.g. Unsworth et al. (2019) Journal of Experimental Psychology. Learning, Memory, and Cognition), mental arithmetic, or spatial reasoning (e.g. Nagase et al. (2015) Journal of the Society for Neuroscience, 38(10), 2631-2651) wherein the performance is normalised for individual capacity to perform this task.

In animals, such as rodents, measurement of “motivational performance” is measured through motor tasks such as the forced swim test or cognitive tasks such as social dominance test or operant conditioning. Motivational performance can also be measured in relation to anxiety in tests such as “elevated plus maze” (e.g. Hollis et al. (2018) Neuropharmacology, 138, 245-256) and “open field and novel object” (e.g. Toledo-Rodgriguez and Sandi, (2011) Frontiers in Behavioral Neuroscience, 5, 17).

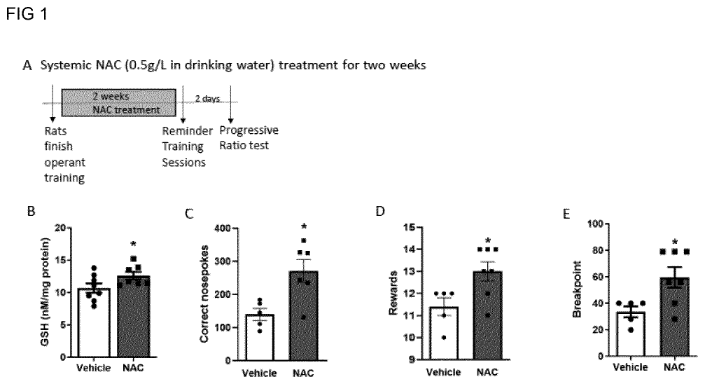

The “nucleus accumbens” is the most ventral part of the striatum and is mainly connected to the limbic system. As a functionally central structure between amygdala, basal ganglia, mesolimbic dopaminergic regions, mediodorsal thalamus and prefrontal cortex, the nucleus accumbens appears to play a modulative role in the flow of the information from the amygdaloid complex to these regions. Together with the prefrontal cortex and amygdala, nucleus accumbens consists a part of the cerebral circuit which regulates functions associated with effort or motivated performance. It is anatomically located in a unique way to serve emotional and behavioral components of feelings. It is considered as a neural interface between motivation and action, having a key-role in food intake, sexual behavior, reward-motivated behavior, stress-related behavior and substance-dependence (Mavridis, Psychiatriki. 2015 October-December; 25(4):282-94). The present invention has surprisingly found that a higher level of glutathione measured in the nucleus accumbens significantly correlates with improved motivational performance (FIG. 1) while stress as measured by cortisol levels is negatively correlated with levels of glutathione in the nucleus accumbens.

The terms “food,” “beverage”, “food product”, “beverage product”, “food composition” and “beverage composition” mean a product or composition that is intended for ingestion by an individual such as a human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

The composition can be any kind of composition that is suitable for human and/or animal consumption. For example, the composition may be selected from the group consisting of: food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

As used herein, “complete nutrition” contains sufficient types and levels of macronutrients (protein, fats and carbohydrates) and micronutrients to be sufficient to be a sole source of nutrition for the individual to which the composition is administered. Individuals can receive 100% of their nutritional requirements from such complete nutritional compositions.

Administration of the compositions of the invention encompass “enteral administration” in all forms, although of oral administration is preferred.

Each of the compounds can be administered at the same time as the other compounds (i.e., as a single unit) or separated by a time interval (i.e., in separate units).

All modes of administration may be considered in combination with glutathione per se.

“Functional derivatives” of compounds of the invention are derived from a similar compound by a chemical reaction. “Functional derivatives” can be formed from the same precursor compound and may be administered to increase glutathione levels in the brain.

A “kit” means that the components of the kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof.

### Embodiments

In several embodiments of the invention, a composition comprising at least one N-acteylcystein or functional derivative and at least one B Vitamin.

The composition is for use in increasing glutathione levels in the brain and/or increasing motivational performance or mental energy in an individual. Such composition increases glutathione in the nucleus accumbens region of the brain to provide the benefits to the subject.

In a further embodiment, the B Vitamins comprises at least one of pyridoxine and/or Vitamin B12.

In a preferred embodiment, a composition of the invention is administered orally.

In a preferred embodiment, a composition is a food supplement or dietary supplement.

In a preferred embodiment, a composition is in the form of a tablet, a gummy or a powder sachet, for example.

In a preferred embodiment, a composition of the invention is in the form of an effervescent tablet.

In several embodiments of the invention, the composition is provided as a kit with a Digital tool, such as a companion App.

In several embodiments of the invention, a composition of the invention is used by healthy individuals in need of i) increasing motivational performance and/or mental energy, ii) increasing cognitive performance, iii) increasing productivity.

In several embodiments of the invention, a composition of the invention is used in decreasing performance anxiety and/or stress.

### Glutathione

Glutathione (GSH) is the most abundant intracellular component of overall antioxidant defenses. GSH, a tripeptide, is synthesized from precursor amino-acids: glycine, cysteine and glutamate in two steps catalyzed by glutamate cysteine ligase (GCL, also known as gamma-glutamylcysteine synthetase, EC 6.3.2.2) and gamma-L-glutamyl-L-cysteine:glycine ligase (also known as glutathione synthetase, EC 6.3.2.3), and GSH synthesis occurs de novo in cells.

Glutathione is also known as Gamma-Glutamylcysteinylglycine, Gamma-L-Glutamyl-L-Cysteinylglycine, Gamma-L-Glutamyl-L-Cystéinylglycine, Glutathion, Glutatión, L-Gamma-Glutamyl-L-Cysteinyl-Glycine, L-Gamma-Glutamyl-L-Cystéinyl-Glycine, L-Glutathion, L-Glutathione, GSH, N-(N-L-gamma-Glutamyl-L-cysteinyl)glycine. It is typically administered as S-acetyl glutathione or reduced L-glutathione.

Glutathione-rich food include: cruciferous vegetables, for example, broccoli, cauliflower, Brussels sprouts, and bok choy; allium vegetables, for example, garlic and onions; eggs, nuts, legumes, lean protein, such as fish, and chicken as well as whey protein. Glutathione-rich herbs include: for example, milk thistle, flaxseed, guso seaweed. Compositions and methods of the invention can also be used in combination with dietary recommendations for a glutathione-rich food to complement the diet.

Lifestyle parameters may affect levels of glutathione in the brain. For example, glutathione is also negatively affected by insomnia. Therefore, compositions and methods of the invention would also include the recommendation to have sufficient sleep.

Psychogenic stress is defined as a state of imminent or perceived threat to homeostasis, where the brain and body invoke various physiological responses to adapt. Glutathione levels in the brain may be affected by such stress.

### Cysteine and N-Acetylcysteine

Cysteine is a non-essential sulfur-containing amino acid important for protein synthesis, detoxification, and diverse metabolic functions. It is required for protein synthesis and for the synthesis of non-protein compounds including taurine, sulfate, coenzyme A, and GSH.

Cysteine itself is a powerful antioxidant and has the potential to trap ROS. Due to the fact that cysteine tends to be absorbed into cells where it cannot exhibit its antioxidant property, N-acetyl cysteine (NAC) is often used in supplement form instead for this purpose.

The N-acetylcysteine or functional derivative thereof can be administered in an amount of about 0.1-100 milligram (mg) of N-acetylcysteine (NAC) or functional derivative thereof per kilogram (kg) of body weight of the subject. In some embodiments, these amounts are provided at least partially by a dipeptide comprising both the N-acetylcysteine or functional derivative thereof and the glycine or functional derivative thereof.

In a particular non-limiting example, the daily doses for a 60 kg subject can be 6 to 6,000 mg/day of NAC or derivative thereof.

### B Vitamins

One or more B vitamins are used in the composition. Non-limiting examples of suitable B vitamins include Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements) and combinations thereof. “Vitamin” includes such compounds obtained naturally from plant and animal foods or synthetically made, pro-vitamins, derivatives thereof, and analogs thereof.

### Pyridoxine

Pyridoxine is the 4-methanol form of vitamin B6, an important water-soluble vitamin that is naturally present in many foods.

In an embodiment, vitamin B6 can include one or more of the following: pyridoxine (PN), pyridoxal 5′-phosphate (PLP), pyridoxine 5′-phosphate (P5P), pyridoxal (PL), pyridoxamine (PM), pyridoxamine 5′-phosphate (PMP), 4-pyridoxic acid, and pyritinol. In a preferred embodiment, at least a portion of any vitamin B6 is PN. At least a portion of the vitamin B6 can be PLP. Absorbed pyridoxamine is converted to PMP by pyridoxal kinase, which is further converted to PLP by pyridoxamine-phosphate transaminase or pyridoxine 5′-phosphate oxidase which also catalyzes the conversion of PNP to PLP.[2] Pyridoxine 5′-phosphate oxidase is dependent on flavin mononucleotide (FMN) as a cofactor produced from riboflavin (vitamin B2).

In an embodiment, Vitamin B6 can be administered in an amount of vitamin B6 in a daily dosage of about 1.0-600 mg vitamin B6, for example about 1.0-200 mg vitamin B6, for example about 1.0-25.0 mg vitamin B6, for example about 10-20 mg of Vitamin B6/day.

A daily dose of the composition can provide 0.1 to 50 times the recommended daily requirement (RDA) of Vitamin B12 per day, more preferably 0.1 to 40 times the recommended daily requirement (RDA) of Vitamin B12 Such dosages may preferably include a daily dose of about 10, 20, 30, or 40 times the RDA of the Vitamin B12 per day. Preferably, the daily dose provides 10 to 40, more preferably 10 to 30 or even more preferably 10 to 25 times the RDA of the Vitamin B12 per day, most preferably about 12 to 21 times the RDA of the Vitamin B12 per day. Further in this regard, the United States RDA of Vitamin B12 is 2.4 micrograms daily for humans of age 14 years and older, so such individuals may be administered a daily dose of the composition that provides about 0.002 mg to about 0.4 mg of Vitamin B12 per day, preferably 0.02 to 0.07 mg of Vitamin B12 per day, more preferably 0.03 to 0.05 mg of Vitamin B12 per day.

One or more other vitamins are used in the composition. Non-limiting examples of suitable other vitamins include vitamin A, Vitamin C, Vitamin D, Vitamin E, Vitamin K, folic acid and biotin), and combinations thereof. “Vitamin” includes such compounds obtained naturally from plant and animal foods or synthetically made, pro-vitamins, derivatives thereof, and analogs thereof.

### Composition Formulations

In one embodiment, the compositions are food compositions, including human and pet food compositions. In several embodiments, the food composition is a product with at least one nutrient for improving motivation performance or mental energy.

For pet food compositions, they may supply the necessary dietary requirements for an animal, animal treats (e.g., biscuits), or dietary supplements. The compositions may be a dry composition (e.g., kibble), semi-moist composition, wet composition, or any mixture thereof. In another embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement is to be administered to the animal in small amounts, or in the alternative, can be diluted before administration to an animal. The dietary supplement may require admixing or can be admixed with water or other diluent prior to administration to the animal.

In another embodiment, the composition is in the form of a food supplement or dietary supplement, in particular in the form of a tablet, a powder sachet or a gummy.

In a preferred embodiment, the composition is in the form of an effervescent tablet.

The effervescent tablets can be produced and controlled same as conventional tablets. These controls include physicochemical properties such as hardness, weight variation, friability, solution time, pH and content uniformity. The effervescent tablets can be produced by a direct compression method, a fusion method, a wet or dry granulation method, or any other suitable method. Low relative humidity (e.g., maximum of 25% or less) and moderate to cool temperatures (e.g., about 25° C. or 77° F.) in the environment may be essential to prevent sticking granule or tablets to the tablet press machine.

In the direct compression method, the effervescent tablet can be formed by compressing the ingredients in the form of powders into a dense mass, for example, by a tablet press machine. The powdered ingredients may be first granulized to similar or equal sizes before being made into tablets, so that the mixtures of powder have excellent flowability without particles segregation. Granulating may not be required if the raw materials are selected to achieve a free-flowing, non-segregating, compressible powder blend. The tablets can then be dried by heat, such as in an oven with air circulation, at a suitable temperature for a suitable time and after cooling can be packed in a suitable package.

In the fusion method, the ingredients can be mixed in a suitable mixer, such as a blender, for an appropriate time. Then, the obtained mixture can be heated to a suitable temperature. The powder may be mixed regularly until the crystallization water of citric acid is released as binder factor (e.g., approximately 30 minutes) and an appropriate pasty mass is obtained. This wet mass can be passed through a sieve to obtain the desired granules, which can then be dried at a suitable temperature for an appropriate time. After drying, the granules can be passed through the sieve again. Other ingredients can be added to the granule mass and mixed for a suitable time. The granule mixtures then can be compressed into tablets by the tablet press machine. Finally, the tablets can be dried and packed in a suitable package.

In the wet granulation method, the ingredients may be milled by a miller, either separately or as a mixture with ethanol, ethanol-water mixture, isopropanol, etc., and the obtained powder can be passed through a sieve and then blended. A binder solution can be added to the mixture to form a pasty mass. This pasty mass can then be passed through a sieve to obtain desired granules, which can then dried. The dried mass can be passed through a sieve again, and other ingredients can be added and mixed. The obtained granule mixtures then can be compressed into tablets by the tablet press machine. Finally, the tablets can be dried and packed in a suitable package. Wet granulation can also be performed by carefully adding 0.1 to 1.0 percent water (weight-to-weight basis) to a blend of raw materials that possess the uniformity, compressibility, and flowability needed to produce good-quality tablets, but which lacks the needed binding properties. The free water which is usually added in the form of a fine spray to selected formulation components while mixing in a suitable blender acts as a binder.

The granulation steps must be precisely timed and the ingredients mixed thoroughly to distribute the granulating fluid evenly in the blend. The mix is then quickly discharged to drying ovens. After drying, the granules are sized, and a final mix is performed. The granules are then compressed into tablets using tablet machines.

The dry granulation method can use special processing equipment known as a “roller compactor” or “chilsonator.” These machines compress premixed powders between two counterrotating rollers under extreme pressure. Depending on the configuration of the roller, the feed material may be compacted into dense ribbon-like materials known as flakes (smooth rolls) or dense briquettes (almond or stick-shaped) if the rollers have grooved or etched surfaces. The compressed material is reduced to the proper size for tablet granulation purposes. Another dry granulation procedure is slugging, in which the powder particles are compressed into large flat tablets or pellets using a tablet press or more usually, a heavy-duty tablet-compacting equipment. The resulting tablet or slug are milled to yield the desired granule characteristics.

The effervescent tablet can be made in any shape and can have any suitable size. As non-limiting examples, the tablet can be 5 mm to 20 mm long, thick, and/or in diameter if having a round shape. The size of the tablet can be from 5 mm to 10 mm, 5 mm to 15 mm, 10 mm to 15 mm, 10 mm to 20 mm, or 15 mm to 20 mm.

The effervescent tablet may comprise a binder, such as polyvinylpyrolidone (PVP) or any other suitable binder. The binder is preferably water-soluble. It can be added as dry powder or in a wet form as an aqueous or hydroalcoholic solution. Mannitol, PEG 6000 and water in small amounts can also be used as a binder. PEG 6000 at 3% use level can be used as a dry binder. The ideal amount of binder is one that makes the tablet hard enough to handle but soft enough to disintegrate and dry enough to be stable. The effervescent tablet may also be formulated without a binder.

### Beverage Compositions

In one embodiment, the compositions are beverage compositions. Such beverage compositions are meant to be consumed by a human or animal. In several embodiments, the beverage is a milk-based beverage; a performance nutrition product, a medical nutrition product; a milk product, e.g. a milk drink, a product with at least one nutrient for improving motivation performance or mental energy.

### Dairy Product

In one embodiment, the composition can be formulated as a “dairy product” together with milk proteins, e.g., milk protein concentrate or milk protein isolate; caseinates or casein, e.g., micellar casein concentrate or micellar casein isolate; or whey protein, e.g., whey protein concentrate or whey protein isolate. Additionally, or alternatively, at least a portion of the protein can be plant protein such as one or more of soy protein, pea protein or canola protein.

### Nutritional Supplement

In one embodiment, the composition of the invention can be formulated as a “nutritional supplement” together with glutathione enhancing compounds of the invention. The compounds of the invention can be used alone or in combination with appropriate additives to make tablets, gummies, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

### Administration

The composition of the invention can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the condition remain. In specific embodiments, the administration occurs until a detectable improvement of at least one symptom occurs and, in further cases, continues to remain ameliorated.

The ideal duration of the administration of the composition can be determined by those of skill in the art.

### Digital Tool/Companion App

In a preferred embodiment, the composition is provided as a kit with a Companion App.

In some embodiments, the composition or kit helps the individual to achieve objectives and/or to support his motivation; reduces his perception of effort to reach his objectives. Such objectives may include any global objectives or daily objectives related to pre-defined goals embodied within the app, such as lifestyle changes, including weight-loss, healthy eating, physical exercise or productivity at work or in private life, as well as any other user-defined goals.

In some embodiments, the companion app presents a onboarding questionnaire, displayed on first launch, to determine consumer wishes and concerns.

In some embodiments, the companion app tracks the progress towards the objectives, as well as activities (physical activity, sleep duration, etc.), optionally through digital tools (connected watch, etc).

In some embodiments, the companion app allows the user to indicate whether or not they have consumed the product.

In some embodiments, the companion app allows the user to capture their mood each day, and visualize the mood together with daily goals in a graph or timeline format.

In some embodiments, the companion app provides rewards in the form of digital trophies, kudo points, or discount vouchers.

Those skilled in the relevant art will appreciate which technology, logic and algorithm can be practiced to program, configure or construct the companion app or another equivalent digital tracking tool.

### EXAMPLES

Enhancement of Glutathione (GSH) Via Systemic Treatment with N-Acetyl Cysteine (NAC) Results in Increased Motivational Performance in Adult Male Rats

Adult male outbred rats were trained for nose poke for saccharine food pellets for one week on an FR1 training schedule as described in the methods below. Rats were then treated with either normal drinking water (vehicle) or N-acetyl cysteine (NAC) in the drinking water (FIG. 1A) at a dose that was previously shown to enhance GSH levels (FIG. 1B). Following 2 weeks of treatment, rats were given an additional 2 training sessions, followed 24 h later by a progressive ratio session designed to test their motivated behaviour. During this progressive ratio session, rats have to increasingly work harder to earn the saccharine pellet, as described in the methods below. NAC-treated rats made significantly more nose pokes during this session (FIG. 1C) and received a greater number of rewards (FIG. 1D). Finally, NAC-treated rats exhibited a significantly higher breakpoint level compared to vehicle-treated counterparts (FIG. 1E). The breakpoint is defined as the last step in the session where the animals received a reward and is a direct correlate of their willingness to exert an effort. A higher breakpoint indicates that the animal exerted greater effort during the session.

### Materials and Methods

Animals: Adult male Wistar rats (Charles Rivers, Saint-German-Nuelle, France) weighing 250-275 gr at the beginning of the experiment were used for all experiments. Rats were individually housed in cages in housing colonies on a 12:12 h reversed light:dark cycle with lights on at 20:00, and lights-off at 8:00. Food and water were available adlibitum. Following a week of acclimatization to the animal facilities, rats were handled for 2 min per day for three days prior to the start of the experiments, in order to habituate to the experimenters.

Operant conditioning: Ten days after introduction to the reversed day-night cycle, rats started training in a fixed ratio 1 reinforcement schedule (FR1). Operant chambers (Coulbourn Instruments, Holliston, MA, US), placed in sound attenuating cubicles, were equipped with a grid, underneath which a tray with standard bedding material was placed for collection of feces and urine after each training session. Each chamber had one food tray and two ports placed on either side of the tray. A cue light was placed in each port and the food tray, whereas a house light was placed above the food tray. The right-hand side port of each chamber was designated as “active”, meaning that spontaneous nosepoking would result in the drop of one 45 mg food pellet (Bio-Serv, Flemington, NJ, USA) to the food tray. Upon nosepoking in the active port, the cue and house lights were turned off, while the tray light turned on and the pellet dropped to the food tray. The two ports remained inactive for 20 s, during which nosepokes would not result in the delivery of a new pellet. Subsequently, the chamber returned to its initial condition. Each training session lasted maximally two hours or until a rat acquired 100 pellets. Each rat received six training sessions (one training on each day for five consecutive days, followed by two days without training and one training session on day 8). Only rats that finished at least two training sessions acquiring 100 pellets before the two-hour mark were used for progressive ratio experiments.

Subsequently, rats were treated with N-acetyl cysteine in the drinking water (500 mg/L) or continued having access to normal water (control). After two weeks of treatment, rats were exposed to another two days of FR1 training to ensure their training performance was similar to pre-treatment levels.

To test motivated behaviour, rats were exposed to a progressive ratio reinforcement schedule (progressive ratio test). Progressive ratio sessions were identical to training sessions except that the operant requirement in each trial (T) was the integer (rounded down) of the function 1.4^{(T-1)} starting at one nosepoke for the first three trials and increasing in subsequent trials for rats (Wanat et al. Nat Neurosci. 2013; 16(4):383-5). Progressive ratio sessions lasted two hours. Correct nosepokes (i.e. nosepokes in the active port and outside the timeout period, thus resulting in food delivery) were calculated to evaluate behavioural performance, as well as the number of acquired rewards and the last completed ratio (breakpoint).

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. Composition comprising at least one N-acetylcysteine or functional derivative and at least one B Vitamin.

2. A method for increasing glutathione levels in the brain and/or increasing motivational performance or mental energy in an individual comprising administering a composition comprising at least one N-acetylcysteine or functional derivative and at least one B Vitamin to the individual.

3. Composition according to claim 1 wherein the B Vitamin comprises at least one of pyridoxine and/or Vitamin B12.

4. Method according to claim 2, wherein said composition is administered orally.

5. Composition according to claim 1, wherein said composition is a food supplement or dietary supplement.

6. Method according to claim 2, wherein the composition is in a form selected from the group consisting of a tablet, a gummy or powder sachet.

7. Method according to claim 6, wherein the tablet is effervescent tablet.

8. Method according to claim 2, wherein the composition is provided as a kit together with a digital tool.

9. Method according to claim 2, to help the individual achieve his objectives and/or to support his motivation; or to reduce his perception of effort to reach his objectives.

10. Method according to claim 2, wherein the individual is a healthy individual in need of at least one of i) increasing motivational performance and/or mental energy; ii) increasing cognitive performance, and iii) increasing productivity.

11. Method according to claim 2 for use in decreasing performance anxiety and/or stress in the individual.
